(19) United States
(12) Patent Application Publication
(10) Pub. No.: US 2025/0387593 A1
(43) Pub. Date: Dec. 25, 2025

(54) Urological Catheter Placement Device

(71) Applicant: Robert R. Kester, Fort Lauderdale, FL (US)

(72) Inventor: Robert R. Kester, Fort Lauderdale, FL (US)

(21) Appl. No.: 19/308,219

(22) Filed: Aug. 23, 2025

Related U.S. Application Data
(60) Provisional application No. 63/729,773, filed on Dec. 9, 2024.

(51) Int. Cl. A61M 25/00; A61M 25/09; A61M 39/10; A61M 39/20

(52) U.S. Cl. CPC A61M 25/0017; A61M 25/09; A61M 39/10; A61M 39/20

(57) ABSTRACT

A urological catheter Device assists inserting a Foley catheter in a patient suffering from Difficult Urethral Catheterization. The urological catheter Device additionally provides a method for detecting when a guidewire fails to pass an obstruction in the urethra. The urological catheter Device includes a Guidewire-chamber. A Foley-catheter Connector is connected to a distal end of the Guidewire-chamber. An Endcap is removably disposed on a proximal end of the Guidewire-chamber. The Guidewire-chamber has a Guidewire-chamber formed therein. A Guidewire can be threaded through the Endcap, the Guidewire-chamber, and the Foley-catheter Connector. A Manipulator-slide travels distally and proximally within the Guidewire-chamber. The Manipulator-slide can be secured to the Guidewire. The Guidewire-chamber has a Guidewire-chamber Slot formed therein and running along the length of the Guidewire-chamber. A Manipulator is located outside the Guidewire-chamber and is connected to the Manipulator-slide via the Guidewire-chamber Slot.

### CROSS-REFERENCE TO RELATED APPLICATIONS

This application claims the benefit of U.S. Provisional Application No. 63/729,773, filed Dec. 9, 2024, which is incorporated herein by reference, including its specification.

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

The Invention relates to urological catheters and, more specifically, to devices for inserting urological catheters.

### Description of the Related Art

In clinical cases, Difficult Urethral Catheterization (DUC) typically first presents in the emergency department. The index patient, an adult male in acute urinary retention, presents to the emergency department with inability to urinate. Standard measures typically begin with attempting to pass a 16-Fr standard Foley catheter (French: 3 Fr =1 mm diameter). In most cases, the Foley catheter passes obstructions in the urethra without difficulty. The inserted Foley catheter carries urine from a bladder of the patient, thereby relieving the patient of acute urinary retention.

Difficult Urethral Catheterization (DUC) in a male adult patient is characterized by the inability to insert a urethral Foley catheter due to an obstruction within the urethra. A urethral Foley catheter, which may be referred to as a “Foley” or “Foley catheter” has a tip with side holes for drainage. The side holes are formed in the tip. The tip is distal to a traction balloon.

Bugeja, S., et al. “A new urethral catheterization device (UCD) to manage difficult urethral catheterization.” World Journal of Urology, vol. 37, no. 4, 24 Sep. 2018, pp. 595-600, https://doi.org/10.1007/s00345-018-2499-9, (hereinafter, “Bugeja et al.”) reported that in a prospective study of seventy-four (74) men, ninety-three percent (93%) of male Foleys passed without complication using their standard Foley insertion protocol, whereas seven percent (7%) had Difficult Urethral Catheterization (DUC), with inability to catheterize the urethra, requiring suprapubic tube insertion in three percent (3%), and gross blood in four percent (4%). In a separate, retrospective review of one hundred twenty-seven (127) other cases at the same institution, two and seven tenths' percent (2.7%) required suprapubic catheter placement for inability to catheterize in male patients with DUC. Therefore, Bugeja et al. teaches to expect seven percent (7%), or one in fourteen, as a realistic percentage of DUC cases to occur in Foley catheter attempts in male urinary retention patients presenting worldwide, with three percent (3%) needing suprapubic catheter placement into the patient's bladder for urinary drainage as a result.

Currently, there are no accepted practice guidelines as to how best to proceed in DUC cases. In current emergency department practice worldwide, typically, the bedside emergency department nurse first will attempt to pass the Foley catheter, most often a 16-Fr size. In cases of DUC, the nurse may make more than one attempt at passage of Foley catheter, with each ending with failure. Thereafter, the charge nurse may attempt to pass the Foley catheter, with comparable results: failure. Finally, the emergency department physician may attempt to pass the Foley catheter with the same outcome: failure. Not uncommonly during these proceedings that may include multiple, failed attempts at Foley passage, blood is seen from the urethra, due to inadvertent injury of the male urethra.

According to Bugeja et al., the potential “incidence-of-ED-use” of the invention might be at a minimum, 7% (See 1. FIG. 3, Page 598.1), and at a maximum, 100%, because the presence of DUC is unknown until a Foley attempt occurs with finding of non-success on initial attempt at catheterizing an adult male in urinary retention.

Villanueva, Carlos, and George P. Hemstreet III. “Difficult Male Urethral Catheterization: A Review of Different Approaches.” International Braz J Urol, vol. 34, no. 4, August 2008, pp. 401-412, https://doi.org/10.1590/s1677-55382008000400002, (hereinafter, “Villanueva et al.”) reported that, in a meta-analysis of three (3) published studies (pooled patient n=87) evaluating the causation of unsuccessful urethral catheter attempts in adult males in urinary retention, the authors determined that urethral stricture is the most common cause of Difficult Urethral Catheterization (DUC), occurring in forty-seven percent (47%) of cases with inability to pass the Foley catheter per urethra.

Moreover, urethral strictures represent fragile tissues, and are prone not only to bleeding when injured, but also perforation and the creation of false urethral passages during repetitive, unsuccessful Foley catheter attempts, with such inadvertent injury generally rendering further successful Foley passage impossible by emergency department staff. In such cases, at some point, the emergency department physician may decide to consult the urologist for assistance.

In addition to emergency-room non-success in standard Foley catheter placement, non-success in the admitted hospital patient bedside, or operating room Foley placement attempts that might require immediate percutaneous or surgical suprapubic catheter placement, as a temporizing measure until such time that urethral healing and further evaluation is completed. It should be noted that urethral strictures may recur following urethral-stricture disruption (or even manipulation, such as a planned urethral dilation) with a “vengeance,” with significantly more potential for ensuing higher grades of cicatrization, and in worst-case scenarios this may require a major operative repair utilizing a buccal mucosal onlay urethroplasty procedure.

BRIEF SUMMARY OF THE INVENTION

An object of the Invention is to provide a urological catheter Device for urethral Foley catheter insertion in adult male patients in acute urinary retention with Difficult Urethral Catheterization (DUC) and a method for urinary catheterizing a patient with DUC that overcomes the disadvantages of the devices and methods of this general type and of the prior art.

A further object of the Invention is to enable passage of a Foley catheter in cases of urethral stricture disease and other causes of DUC.

A further object of the Invention is to circumvent problems of Difficult Urethral Catheterization (DUC) in the adult male with acute urinary retention, and to prevent inadvertent emergency department (ED) staff injuries to the male urethra from repetitive and failed attempts in the emergency department when using standard techniques.

A further object of the Invention is to provide a Device that can be used by emergency department physicians at bedside to pass a Foley catheter in cases of DUC caused by urethral stricture disease, as well as other causes of DUC.

A further object of the Invention is to provide a Device to enable passage of a Foley catheter in patients with the following conditions: benign prostatic hyperplasia (BPH), bladder neck contracture (after Transurethral Resection of the Prostate (TURP); or postop to radical (robotic or open operative) prostatectomy for prostate cancer), false passage and prostate cancer (per se).

In accordance with the objects of the Invention, a Device is configured for use with a Guidewire in conjunction with a recommended Council-tip Foley (or other related tools). A Guidewire-chamber facilitates a Guidewire to be captured at its proximal end by a Manipulator-slide. The Guidewire-chamber can include a Guidewire-chamber Wall with a Guidewire-chamber Slot formed therein. The Guidewire-chamber Slot runs longitudinally over nearly, but not entirely, the length of the Guidewire-chamber Wall, from the proximal end to the distal end of the Guidewire-chamber, with the Guidewire-chamber Slot to the end at its proximal aspect. The Guidewire-chamber can be tubular or otherwise shaped. The Manipulator-slide can attach to a Manipulator. In use, the Manipulator-slide traverses freely inside the Guidewire-chamber both distally and proximally along the length of the Guidewire-chamber. An attending physician or other provider can manipulate the Snap-on Manipulator-slide by using the Handle-post and attached thumb-ring. The distal end of the Guidewire-chamber preferably has affixed a Foley-catheter Connector, and the proximal end of the Guidewire-chamber preferably has a removable Endcap with an incorporated Endcap Luer-lock. The Endcap Luer-lock allows for insertion and attachment of additional tools such additional Guidewires, other tools, or fluid injection. The removal of the Endcap allows for extraction of the Guidewire and Manipulator-slide, facilitating exchange of either or both, or the introduction of other related instrument tools.

In accordance with the objects of the Invention, a method for determining when to use the urological catheter Device is provided. The urological catheter Device should be used after attempting and failing a “one-time gentle urethral catheterization,” typically performed at bedside, that fails to pass the standard Foley catheter. Use of the Device can indicate an adult male patient with an impassable urethral obstruction, in which device placement is anticipated to occur most often in the emergency department of any hospital worldwide, in the operating room, and elsewhere when DUC might be encountered.

In accordance with the objects of the Invention, guidelines have been developed for use of the urological catheter Device for management of emergency department cases of Difficult Urethral Catheterization in the adult male with acute urinary retention. Perhaps, with proper “teaching” of the Invention's benefit, emergency department protocol could be enhanced by first use of the Invention on all cases of adult male urinary retention, due to improvement of outcome by using the Invention to help prevent accidental or inadvertent injury or piercing of the urethral wall during attempts at Foley catheter insertion. In addition, the safety feature as a “Serpentinization” phenomenon is caused by impassable urethral obstruction, thereby defining patients indicated for urgent temporary bladder drainage by percutaneous suprapubic tube insertion.

A further object of the Invention is to provide a urological catheter Device that may be applied, not only to cases of urethral stricture disease, but also to other cases involving urethral obstruction, particularly in cases of DUC caused by benign prostatic hyperplasia (BPH). In urethral stricture disease, generally, the smallest internal diameter of obstruction caused by DUC is 2-4 Fr. As stated, the urological catheter device also may be applied in all other causes of urethral obstruction, particularly in cases of DUC caused by benign prostatic hyperplasia (BPH). BPH is now the likely first-most common cause of DUC in the current contemporary era of treatment using “alpha-blocker-medication-as-primary-treatment” for BPH patients (in the observations of the Inventor). Rationale: alpha-blockers (e.g., FLOMAX®, etc.) enable continued prostate gland growth, enabling urethral obstruction vis-à-vis angulation of prostatic urethra (colloquial term is “prostate shelf”). Frendl, et. al. “Early vs Delayed Transurethral Surgery in Acute Urinary Retention: Does Timing Make a Difference?” J Urology, vol 210, September 2023, pp 493-499, https://doi.org/10.1097/JU.0000000000003559 (hereinafter, Frendl et al.”), recount that acute urinary retention is a complication of the obstructive process often caused by benign prostatic hyperplasia (BPH), one of the most common diseases in older men. Frendl et al. reference that the overall incidence of BPH-related acute urinary retention is 4.5-6.8 per 1,000 men per year, with over 1 in 10 men in their 70s and over 1 in 3 men in their 80s experiencing an episode of urinary retention. Furthermore is noted that research from California suggests that the incidence BPH-related acute urinary retention is increasing.

In accordance with the objects of the Invention, a urological catheter Device (which is sometimes referred to as “The Device” in this specification) is provided for urethral Foley catheter insertion in adult male patients in acute urinary retention with Difficult Urethral Catheterization (DUC). The devices and methods of the Invention help prevent accidental or inadvertent injury or piercing of the urethral wall during attempts at Foley catheter insertion. This Invention is intended for use in combination with, but not limited to, a urinary-tract Guidewire and urethral Council-tip Foley catheter, with a hole at the distal-most tip.

The Device avoids urethral obstruction during clinical cases of Difficult Urethral Catheterization (DUC) in a male adult patient, in which the standard attempt at catheterization by way of a Urethral Foley Catheter (standard “Foley,” with side holes for drainage at its tip, distal to the traction balloon) is met with impassable result. The Device is intended to be used in (but is not limited to) the passing of a Foley in cases of DUC. The Device places a “Guidewire” or another related instrument (as the Device is not necessarily confined to Guidewire passage only) beyond the urethral obstruction. When used to pass a Council-tip Foley catheter over a Guidewire that is passed beyond the urethral obstruction, the Council-tip Foley pre-positioned on the Foley-catheter Connector (with additional hole at axial tip designed for passage of Council-tip Foley over a Guidewire) may be passed over the Guidewire and into urinary bladder.

The Device can include an important “safety feature.” That is, the Device can include an “indicator” of impassable urethral obstruction. “Serpentinization” has been noted on initial ex-vivo testing of mock-up Devices. Serpentinization occurs when the floppy tip of the Guidewire passed into the male urethra is unable to traverse the urethral stricture (or other urethral obstruction). Upon meeting an impassable urethral obstruction, the Guidewire “serpentines,” visibly seen as a Guidewire “sinewave” configuration in the Guidewire-chamber (made of transparent-to-opaque medical-grade material, which may be shaped as “tubular” or other configuration), the “Serpentinization” is associated with non-advancement of the Guidewire. (Note: the “Guidewire-chamber,” is defined later in this specification.) Such a circumstance will lead the emergency physician (or other practitioner) to immediately consult urology, for the express indication of urgent placement of a suprapubic catheter to achieve bladder drainage.

In accordance with the objects of the Invention, a Manipulator-slide is disposed on the Guidewire. Examples of suitable Manipulator-slides include fluted, splined, grooved and other shaped Manipulator-slides. The Manipulator-slide can be a Snap-on Manipulator-slide. A particularly useful example of a Snap-on Manipulator-slide can be formed as a clamshell, which is closed over the stiff proximal end of the Guidewire and clasped in place. Once the Snap-on Manipulator-slide is placed on the Guidewire, the position of the Snap-on Manipulator-slide on the Guidewire will not change in normal use until the physician intentionally releases the Snap-on Manipulator-slide from the Guidewire. Molded or otherwise placed onto the Manipulator-slide can be a Handle-post. A detachable Manipulator Thumb-ring (“thumb-ring”) can be snapped upon the Manipulator post. The Manipulator Thumb-ring may be detached from the Manipulator post during Guidewire passage as desired. The Manipulator-slide is positioned within a transparent tubular Guidewire-chamber (“Guidewire-chamber,” tubular or otherwise shaped, transparent or other), which is slotted from its proximal end, and then remaining slotted until about one centimeter (˜1 cm) from its distal end (solid from there until distal end). A detachable Endcap (“Endcap”), containing a Luer-lock (“Luer-lock”), attaches to the proximal end and may be removed by the physician (or another provider). The endcap can be made as a one piece unibody including the collar and Luer-lock.

The purpose of the Manipulator-slide is to allow the physician (or another practitioner) to smoothly extend and retract the Guidewire (or another related instrument) as necessary. Replacing the Guidewire with another-sized Guidewire (or another related instrument) for insertion into the urethra is allowed by: Removal of the Endcap and then withdrawing in a retrograde fashion the Manipulator-slide; opening the Manipulator-slide Clamshell clasp; wire removal and replacement with new wire (or another related instrument); re-closing the clasp; replacing the Manipulator-slide back into the Guidewire-chamber; and replacing the Endcap. Also, the Guidewire can be passed through the Guidewire-chamber free hand via the Luer-lock. Additional Guidewire(s) may be passed through the Endcap Luer-lock (as well as fluid injections), whereupon the additional Guidewire(s) pass by the Manipulator-slide by virtue of the channels (fluted, splined or grooved) designed into the Manipulator-slide. The distal end of the Guidewire-chamber has affixed a Foley catheter Connector (“connector”), through which the Guidewire (or another related instrument) passes.

The excursion of the Guidewire (or another related instrument) through the connector (i.e., out of, or into the urological catheter Device) is controlled by excursion of the Manipulator-slide, which can be easily used in a back-and-forth (“in-and-out”) manner to great effect, particularly when passing a Guidewire through a narrow-diameter urethral stricture.

While using a Guidewire in the Manipulator-slide, when with Manipulator-slide has progressed maximally towards the distal end of the Guidewire-chamber (and Guidewire confirmed to be positioned within the urinary bladder using appropriate imaging guidance), a pre-positioned Council-tip Foley catheter is then deployed over the Guidewire and into the patient's urinary bladder.

### Spatial Relationships

The size and dimensions of several of the parts can be expressed in terms of a relationship between parts of the Device and between the Device and parts of the patient's anatomy.

The length of the Guidewire-chamber Slot should be at least as long as the maximal length of an adult male from urethral meatus to bladder neck, determined in a medical study of adult male urethral length to be 28 cm. (11 inches), by Ojewola et al., in the medical journal, BMC Urology, 2023; 185:1-7; https://doi.org/10.1186/s12894-023-01360-0). If the distal end of the Guidewire is at the distalmost tip of the Foley catheter when the Guidewire is fully retracted to the proximal slot end, then the distal Guidewire end will reach the bladder of the patient when the Guidewire is extended to the distal slot end. In practice, the length of the slot is variable with consideration of maximal reported male urethral length of 28 cm.

With the foregoing and other objects in view there is provided, in accordance with the Invention, a urological catheter Device for urethral Foley catheter insertion in adult male patients in acute urinary retention with Difficult Urethral Catheterization (DUC) and a method for urinary catheterizing a patient with DUC.

Other features that are considered as characteristic for the Invention are set forth in the appended claims.

Although the Invention is illustrated and described herein as embodied in a urological catheter Device, the Invention should not be limited to the details shown in those embodiments because various modifications and structural changes may be made without departing from the spirit of the Invention while remaining within the scope and range of equivalents of the claims.

The construction and method of operation of the Invention and additional objects and advantages of the Invention is best understood from the following description of specific embodiments when read in connection with the accompanying drawings.

### BRIEF DESCRIPTION OF THE SEVERAL VIEWS OF THE DRAWING

FIG. 1 is a left elevational view of a urological catheter Device according to the Invention.

FIG. 2 is a rear (proximal) sectional view of the urological catheter Device shown in FIG. 1 taken along line A-A in FIG. 1.

FIG. 3 is a top elevational view of the urological catheter Device.

FIG. 4 is a rear (proximal) elevational view of the urological catheter Device.

### DETAILED DESCRIPTION OF THE INVENTION

In the Summary of the Invention above and in the Detailed Description of the Invention, and the claims below, and in the accompanying drawings, reference is made to features including method steps of the Invention. It is to be understood that the disclosure of the Invention in this specification includes all possible combinations of such features. For example, where a particular feature is disclosed in the context of a particular aspect or embodiment of the Invention, or a particular claim, that feature can also be used, to the extent possible, in combination with and/or in the context of other particular aspects and embodiments of the Invention, and in the Invention generally.

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm.

### Urological Catheter Device

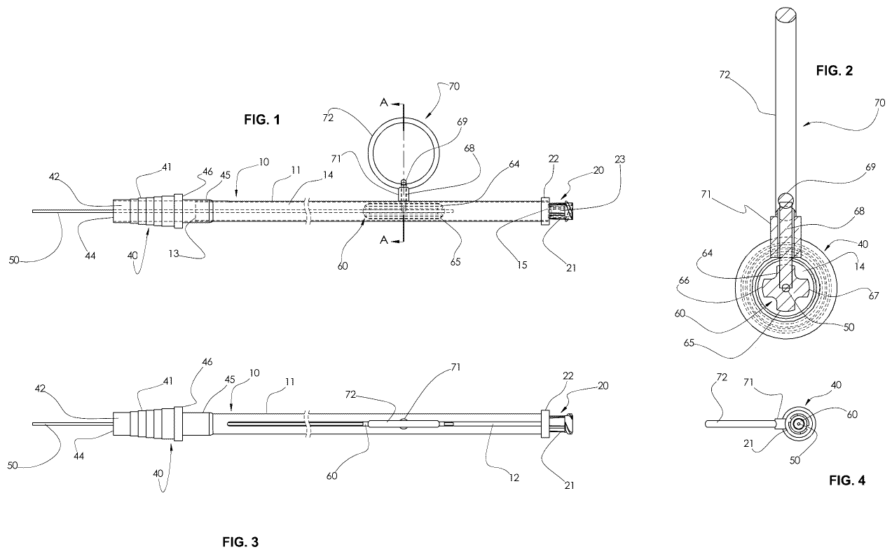

FIGS. 1-4 show a preferred embodiment of the urological catheter Device. The urological catheter Device includes a Guidewire-chamber 10. In the version shown, the Guidewire-chamber 10 is transparent. In versions that are not shown, the Guidewire-chamber 10 can be translucent, tinted, or opaque. In the version shown in FIGS. 1-4, the Guidewire-chamber 10 is tubular with a Guidewire-chamber lumen 14 having an annular cross section. In alternate versions, the Guidewire-chamber 10 can define a Guidewire-chamber lumen 14 with different, non-annular cross sections: for example, triangular, square, polygonal, or elliptical cross sections. In the version shown in FIGS. 1-4, the Guidewire-chamber 10 is rigid. In alternative versions, the Guidewire-chamber 10 can be flexible. The Guidewire-chamber 10 is made of medically acceptable material. The Guidewire-chamber 10 has a distal Guidewire-chamber opening 13 and a proximal Guidewire-chamber opening 15. A Guidewire-chamber wall 11 extends between the distal Guidewire-chamber opening 13 and the proximal Guidewire-chamber opening 15. A Guidewire-chamber Slot 12 is formed in the Guidewire-chamber Wall 11. The Guidewire-chamber Slot 12 runs longitudinally (i.e., between about one centimeter (˜1 cm) proximal to the distal Guidewire-chamber Opening 13 and the Guidewire-chamber Slot 12 continues through the proximal Guidewire-chamber Opening 15).

A Luer-lock Endcap 20 is disposed on the proximal end of the Guidewire-chamber 10. The Luer-lock Endcap 20 has an Endcap Collar 22 and Luer-lock 21. The Endcap Collar 22 is removable and covers the proximal Guidewire-chamber opening 15 and closes the proximal end of the Guidewire-chamber 10. The Luer-lock Endcap 20 is removable from the Guidewire-chamber 10. The Luer-lock Endcap 20 has an Endcap Lumen 23 formed therein. The Endcap Lumen 23 is fluidly connected to the proximal Guidewire-chamber opening 15. An Luer-lock 21 is disposed on the Luer-lock Endcap 20. The Endcap 20 has an Endcap Lumen 23.

A Foley-catheter Connector 40 is disposed on the distal Guidewire-chamber Opening 13. The Foley-catheter Connector 40 can be connected to a proximal end of a Council Foley catheter. The Foley-catheter Connector 40 has a Foley-catheter Connector Wall 41. The Foley-catheter Connector Wall 41 has a frustoconical shape with Foley-catheter Connector steps ending at 42. The Foley-catheter Connector Wall 41 defines a Foley-catheter Connector Lumen 44. The Foley-catheter Connector Lumen 44 is fluidly connected to the Guidewire-chamber Lumen 14. The Guidewire 50 can be extended from the Guidewire chamber 10, through the Foley-catheter Connector 40 via the Foley-catheter Connector Lumen 44, and beyond the distal-most aspect of the Foley-catheter Connector. A proximal end of the Foley-catheter Connector 40 has a Foley-catheter Connector Rear-wall 46 that is connected to a Foley-catheter Connector Skirt 45. The Foley-catheter Connector Skirt 45 fits over distal Guidewire-chamber Opening 13 of the Guidewire-chamber wall 11, where they non-detachably join.

A Manipulator-slide 60 travels longitudinally within the Guidewire-chamber 10. The Manipulator-slide 60 has a Manipulator Post 68 extending dorsally from the Manipulator-slide 60. The Manipulator Post 68 extends through the Guidewire-chamber Slot 12 and is releasably connectable to a Manipulator Assembly 70. The Manipulator Post 68 ends at its distal terminus as a Manipulator Post Ball 69.

A Manipulator Assembly 70 can be moved distally and proximally, which moves the Manipulator slide 60 distally and proximally within the Guidewire Chamber 10, which extends and retracts the Guidewire 50 from the Guidewire Chamber 10. The Manipulator Assembly 70 has a Manipulator Cuff 71. The Manipulator Cuff 71 fits over the Manipulator Post 68. The Manipulator Cuff 71 is secured by the Manipulator Post Ball 69. The Manipulator Assembly 70 can be connected and disconnected to the Manipulator-slide 60. The physician may control extension/retraction maneuvers with his/her thumb through the detachable Thumb-ring 72.

As shown in FIG. 2, the Manipulator-slide 60 has a cruciform cross section and includes four cruciate wings: a dorsal cruciate wing 64, a ventral cruciate wing 65, a left cruciate wing 66, and a right cruciate wing 67. Other quantities of wings are possible. In the version shown, the Cruciate Wings 64-67 are configured as being straight, but the Cruciate Wings 64-67 can be fluted or otherwise. The Manipulator-slide 60 includes a dorsal Manipulator-slide clamshell and a ventral Manipulator-slide clamshell, and a Manipulator-slide Hinge that interconnects the Dorsal Manipulator-slide clamshell and the Ventral Manipulator-slide Clamshell. The dorsal Manipulator slide clamshell and the ventral Manipulator slide clamshell can be separated to insert or remove the Guidewire 50. Once the Guidewire 50 is inserted, the dorsal Manipulator clamshell and the ventral Manipulator clamshell can be closed with a clasp to sandwich the Guidewire 50. The two (2) manipulator-slide clamshell pieces, joined by a hinge and clasped skirt, enable the “Snap-on” feature, thereby releasably securing the Guidewire 50 in place.

The Guidewire 50 is flexible. The Guidewire 50 is held by the Manipulator-slide 60. The Guidewire 50 is sandwiched between the dorsal Manipulator-slide clamshell 61 and ventral Manipulator-slide Clamshell 62. When closed, the position of the Manipulator-slide 60 is fixed on the Guidewire 50, and its movement within the Guidewire Chamber 10 controlled by the physician (or other practitioner) by movement of the Manipulator-slide 60.

### Additional Considerations

Although the present Invention has been described in considerable detail with reference to certain preferred versions thereof, other versions are possible. Therefore, the spirit and scope of the appended claims should not be limited to the description of the preferred versions contained herein.

The reader's attention is directed to all papers and documents which are filed concurrently with this specification, and which are open to public inspection with this specification, and the contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C. § 112, ¶ 6.

What is claimed is:

1. A urological catheter Device for detecting when a stricture within a urethra of a male patient blocks insertion of a Guidewire, comprising:
a Guidewire-chamber for housing, retracting, and extending the Guidewire past the stricture, said Guidewire-chamber having a distal Guidewire-chamber Lumen formed therein for allowing the Guidewire to be extended distally therethrough, said Guidewire-chamber having a proximal Guidewire-chamber Lumen formed therein for allowing the Guidewire to be extended proximally therethrough, said Guidewire-chamber having a Guidewire-chamber Wall running from said distal Guidewire-chamber Lumen to said proximal Guidewire-chamber Lumen, said Guidewire-chamber wall having a Guidewire-chamber Slot being formed therein, said Guidewire-chamber Slot running lengthwise, said Guidewire-chamber Wall defining a Guidewire-chamber lumen interconnecting said distal Guidewire-chamber Lumen and said proximal Guidewire-chamber Lumen.

2. The urological catheter Device according to claim 1, further comprising:
a Foley-catheter Connector for connecting to a proximal end of a Council Foley catheter, said Foley-catheter Connector having a Foley-catheter Connector Wall forming a Foley-catheter Connector Lumen with a proximal Foley-catheter Connector opening and a distal Foley-catheter Connector Opening.

3. The urological catheter Device according to claim 2, wherein said Foley-catheter Connector is shaped generally frustoconically.

4. The urological catheter Device according to claim 3, wherein said Foley-catheter Connector includes steps.

5. The urological catheter Device according to claim 1, further comprising an Endcap with an Endcap Lumen formed therethrough, said Endcap being removably disposed on said Guidewire-chamber with said Endcap Lumen being fluidly connected to said Guidewire-chamber Lumen, said Endcap allowing a Manipulator-slide to be inserted or removed from said Guidewire-chamber when said Endcap is removed, and said Endcap retaining the Manipulator-slide within said Guidewire-chamber when said Endcaps is connected to said Guidewire-chamber.

6. The urological catheter Device according to claim 5, wherein said Endcap includes an Endcap Luer-lock for connecting to a further device.

7. The urological catheter Device according to claim 1, wherein said Guidewire-chamber is transparent.

8. The urological catheter Device according to claim 1, wherein said Guidewire-chamber is translucent.

9. The urological catheter Device according to claim 1, further comprising a Manipulator-slide for extending and retracting the Guidewire into and from said Guidewire-chamber, said Manipulator-slide being sized to travel in said Guidewire-chamber Lumen, said Manipulator-slide being reachable through said Guidewire-chamber Slot.

10. The urological catheter Device according to claim 9, further comprising a Manipulator Post being connected to said Manipulator-slide and extending through said Guidewire-chamber Slot.

11. The urological catheter Device according to claim 10, further comprising a Post knob being disposed on said Manipulator Post and being located outside said Guidewire-chamber.

12. The urological catheter Device according to claim 10, further comprising a Thumb-ring for being held during operation, said Thumb-ring being connected to said Manipulator Post.

13. The urological catheter Device according to claim 9, wherein said Manipulator-slide includes a dorsal Manipulator-slide Clamshell, a ventral Manipulator-slide Clamshell, and a manipulator hinge interconnecting said dorsal Manipulator-slide Clamshell and said ventral Manipulator-slide Clamshell, said dorsal Manipulator-slide Clamshell being separatable from said ventral Manipulator-slide Clamshell to allow the Guidewire to be inserted and released, said dorsal Manipulator-slide Clamshell being closable on said ventral Manipulator-slide Clamshell to hold the Guidewire.

14. The urological catheter Device according to claim 13, wherein said Manipulator-slide is configured to secure to the Guidewire.

15. The urological catheter Device according to claim 9, wherein said Manipulator-slide has a Manipulator-slide Cruciform Cross-section.

16. The urological catheter Device according to claim 2, further comprising a Guidewire for inserting into the urethra, said Guidewire running through said proximal Guidewire-chamber Lumen, and said Foley-catheter Connector.

17. The urological catheter Device according to claim 16, wherein said Guidewire is flexible enough to bend when striking the stricture in the urethra while said Guidewire is being extended, said Guidewire indicating an impassable urethral obstruction when bent.

18. The urological catheter Device according to claim 17, wherein said Guidewire has a diameter between 0.089 cm and 0.096 cm.

19. The urological catheter Device according to claim 2, further comprising:
an Endcap with an Endcap Lumen formed therethrough, said Endcap being removably disposed on said Guidewire-chamber with said Endcap Lumen being fluidly connected to said Guidewire-chamber Lumen, said Endcap allowing a Manipulator-slide to be inserted or removed from said Guidewire-chamber when said Endcap is removed, and said Endcap retaining the Manipulator-slide within said Guidewire-chamber when said Endcaps is connected to said Guidewire-chamber;
a Manipulator-slide for extending and retracting a Guidewire into and from said Guidewire-chamber, said Manipulator-slide being sized to travel in said Guidewire-chamber Lumen, said Manipulator-slide being reachable through said Guidewire-chamber Slot; and
a Guidewire for inserting into the urethra, said Guidewire being secured by said Manipulator-slide, said Guidewire extending through said Guidewire-chamber, and said Foley-catheter Connector.

20. The urological catheter Device according to claim 19, wherein said Manipulator-slide is removable from said Guidewire-chamber when said Endcap is removed from said Guidewire-chamber.

21. The urological catheter Device according to claim 1, wherein said Guidewire-chamber Slot is linear.

22. The urological catheter Device according to claim 1, wherein said Guidewire-chamber Slot runs longitudinally.

23. The urological catheter Device according to claim 1, wherein said Guidewire-chamber is tubular shaped.
